(19) United States
(12) Patent Application Publication
(10) Pub. No.: US 2025/0366755 A1
(43) Pub. Date: Dec. 4, 2025

(54) VOLTAGE-TO-FRQUENCY ELECTROCARIOGRAM MEASUREMENT NODE

(71) Applicant: KONINKLIJKE PHILIPS N.V., EINDHOVEN (NL)

(72) Inventors: RICHARD EARL BROWN, DAVENPORT, FL (US); PAUL FRANZ REDDER, NEWBERRY, FL (US); BRUCE GEOFFREY APPLETON, ORLANDO, FL (US)

(21) Appl. No.: 19/110,190

(22) PCT Filed: Sep. 12, 2023
(86) PCT No.: PCT/EP2023/074994

§ 371 Date: Mar. 10, 2025

Related U.S. Application Data
(60) Provisional application No. 63/406,442, filed on Sep. 14, 2022.

(51) Int. Cl. A61B 5/28; A61B 5/00; A61B 5/301; A61B 5/308; A61B 5/339

(52) U.S. Cl. CPC A61B 5/28; A61B 5/301; A61B 5/308; A61B 5/339; A61B 5/7228; A61B 2562/228

(57) ABSTRACT

A system for acquiring electrocardiogram (ECG) pulses from a subject, comprising: a virtual ground; and a plurality of measurement nodes connectable to a plurality of corresponding ECG electrodes, wherein the plurality of measurement nodes are connected to the virtual ground, and wherein each measurement node comprises: a voltage-to-frequency converter (VFC) configured to convert an ECG signal from the corresponding ECG electrode to a frequency signal; an optical converter configured to convert the frequency signal from the VFC to an optical signal, and to output the optical signal via an output fiber-optic cable; and a DC power converter configured to receive a modulated optical signal via an input fiber-optic cable, to recover DC power from the modulated optical signal, and to supply the DC power to at least the VFC and the optical converter.

### FIELD OF THE DISCLOSURE

The present disclosure is directed generally to systems for acquiring electrocardiogram (ECG) pulses from a subject in an MRI environment.

### BACKGROUND

Electrocardiogram systems monitor functionality of a subject's heart by acquiring and measuring ECG pulses though ECG electrodes placed in contact with the subject. ECG systems may be useful in monitoring subjects in potentially stressful situations during medical diagnostics and treatment. For example, during a magnetic resonance imaging (MRI) procedure, the subject is confined to a relatively small diameter bore of an MRI scanner for an extended period of time, which may cause anxiety. Therefore, ECG electrodes may be attached to the subject while inside the bore during the MRI procedure to provide ECG pulses in real-time, and thus information regarding the subject's well-being.

However, the MRI scanner is a harsh environment for detecting small, millivolt, ECG electrical pulses produced by the heart. Conventional ECG systems are implemented with long electrical ECG leads individually connecting the ECG electrodes to an ECG module, which serves as the analog front end for the ECG electrodes, including amplification and analog-to-digital conversion of the ECG pulses. Indeed, typical MRI compatible multi-electrode ECG measurement setups are implemented with galvanic ECG lead connections between electrodes on a patient and a remotely located module which processes signals of all leads in a single location. The ECG leads are susceptible to MRI noise pickup during active scans resulting in signal degradation. The ECG leads can also be a source of RF heating causing thermal injuries to sedated patients if not placed correctly. Furthermore, ECG equipment inside the MRI bore can interfere with MRI image quality.

### SUMMARY OF THE DISCLOSURE

Accordingly, there is a continued need for systems suitable for acquiring electrocardiogram pulses from a subject in an MRI environment. Various embodiments and implementations herein are directed to a system for acquiring electrocardiogram pulses from a subject. The system includes a plurality of measurement nodes connectable to a plurality of corresponding ECG electrodes, wherein each of the plurality of corresponding ECG electrodes are attachable to the subject, wherein the plurality of measurement nodes are connected to the virtual ground, and wherein each of the plurality of measurement nodes comprises: a voltage-to-frequency converter (VFC), an optical converter, and a DC power converter.

Generally, in one aspect, a system for acquiring ECG pulses from a subject is provided. The system comprises a virtual ground, and a plurality of measurement nodes connectable to a plurality of corresponding ECG electrodes, where each of the plurality of corresponding ECG electrodes are attachable to the subject, and where the plurality of measurement nodes are connected to the virtual ground. Each of the plurality of measurement nodes comprises: a voltage-to-frequency converter (VFC) configured to convert an ECG signal from the corresponding ECG electrode to a frequency signal; an optical converter configured to convert the frequency signal from the VFC to an optical signal, and to output the optical signal via an output fiber-optic cable; and a DC power converter configured to receive a modulated optical signal via an input fiber-optic cable, to recover DC power from the modulated optical signal, and to supply the DC power to at least the VFC and the optical converter.

According to an embodiment, the modulated optical signal comprises an embedded clock signal, and wherein the system further comprises, for each of the plurality of measurement nodes, a clock recovery circuit configured to receive the modulated optical signal with the embedded clock signal via the input fiber-optic cable, to recover the embedded clock signal from the modulated optical signal, and to supply the recovered clock signal to at least the VFC and the optical converter for synchronization.

According to an embodiment, the system further includes an ECG module configured to provide the modulated optical signal to the plurality of measurement nodes via the input fiber-optic cables respectively, to receive the optical signals from the plurality of measurement nodes via the output fiber-optic cables respectively, and to convert the optical signals to the ECG pulses.

According to an embodiment, the plurality of measurement nodes comprise a left arm (LA) measurement node, a right arm (RA) measurement node, and a left leg (LL) measurement node, and a right leg (RL) measurement node.

According to an embodiment, the DC power converter of each measurement node of the plurality of measurement nodes comprises a photovoltaic cell.

According to an embodiment, each measurement node of the plurality of measurement nodes further comprises a programmable gain amplifier (PGA) connected to an input of the VFC, and configured to amplify the ECG signal.

According to an embodiment, the system further includes a monitor configured to display the ECG pulses output by the ECG module.

According to an embodiment, the subject is positioned within a magnet resonance imaging (MRI) bore while the ECG pulses are acquired.

According to an embodiment, the modulated optical signal comprises a pulse width modulated (PWM) optical signal.

According to an embodiment, the modulated optical signal comprises a frequency modulated or amplitude modulated optical signal.

According to an embodiment, the VFC of each measurement node is configured to convert the ECG signal to a different frequency signal relative to every other measurement node.

According to another aspect, a system for acquiring ECG pulses from a subject is provided. The system includes: (i) a virtual ground; (ii) a plurality of measurement nodes connectable to a plurality of corresponding ECG electrodes, wherein each of the plurality of corresponding ECG electrodes are attachable to the subject and comprise a left arm (LA) measurement node, a right arm (RA) measurement node, and a left leg (LL) measurement node, and a right leg (RL) measurement node, wherein the plurality of measurement nodes are connected to the virtual ground, and wherein each of the plurality of measurement nodes comprises: a programmable gain amplifier (PGA) configured to amplify the ECG signal; a voltage-to-frequency converter (VFC) configured to convert an ECG signal to a frequency signal; an optical converter configured to convert the frequency signal from the VFC to an optical signal, and to output the optical signal via an output fiber-optic cable; and a DC power converter configured to receive a modulated optical signal via an input fiber-optic cable, to recover DC power from the modulated optical signal, and to supply the DC power to at least the VFC and the optical converter; (iii) an ECG module configured to provide the modulated optical signal to the plurality of measurement nodes via the input fiber-optic cables respectively, to receive the optical signals from the plurality of measurement nodes via the output fiber-optic cables respectively, and to convert the optical signals to the ECG pulses; and (iv) a monitor configured to display the ECG pulses output by the ECG module.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a schematic representation of a set of measurement nodes for monitoring ECG signals from a subject, in accordance with an embodiment.

FIG. 2 is a schematic representation of an ECG system for monitoring ECG pulses from a subject, implemented within magnetic resonance imaging (MRI) system, in accordance with an embodiment.

FIG. 3 is a schematic representation of a representative measurement node for monitoring ECG signals from a subject, in accordance with an embodiment.

### DETAILED DESCRIPTION OF EMBODIMENTS

The present disclosure describes various embodiments of an electrocardiogram ECG system configured to acquire ECG pulses from a subject. More generally, Applicant has recognized and appreciated that it would be beneficial to provide an ECG system configured to operate within an MRI environment. The ECG system includes a plurality of measurement nodes connectable to a plurality of corresponding ECG electrodes, wherein each of the plurality of corresponding ECG electrodes are attachable to the subject, wherein the plurality of measurement nodes are connected to the virtual ground, and wherein each of the plurality of measurement nodes comprises: a voltage-to-frequency converter (VFC), an optical converter, and a DC power converter. According to an embodiment, the systems described or otherwise envisioned herein can, in some non-limiting embodiments, be implemented as an element for a commercial product for MRI environments.

According to an embodiment, the ECG systems described or otherwise envisioned herein comprise synchronized measurement nodes configured to transmit ECG pulses acquired from a subject through corresponding ECG electrodes attached to a body of the subject. Each measurement node includes all necessary components at the corresponding ECG electrode to which it is attached for formatting the ECG pulses. This eliminates the need for a conductive ECG lead to connect the measurement node to an ECG module. Without conductive ECG leads, the measurement nodes reduce signal degradation otherwise caused by noise within the bore of an MRI system, for example, caused by conventional measurement nodes and ECG modules. The measurement nodes may be snapped or clipped onto existing ECG electrodes, or may incorporate dedicated ECG electrodes.

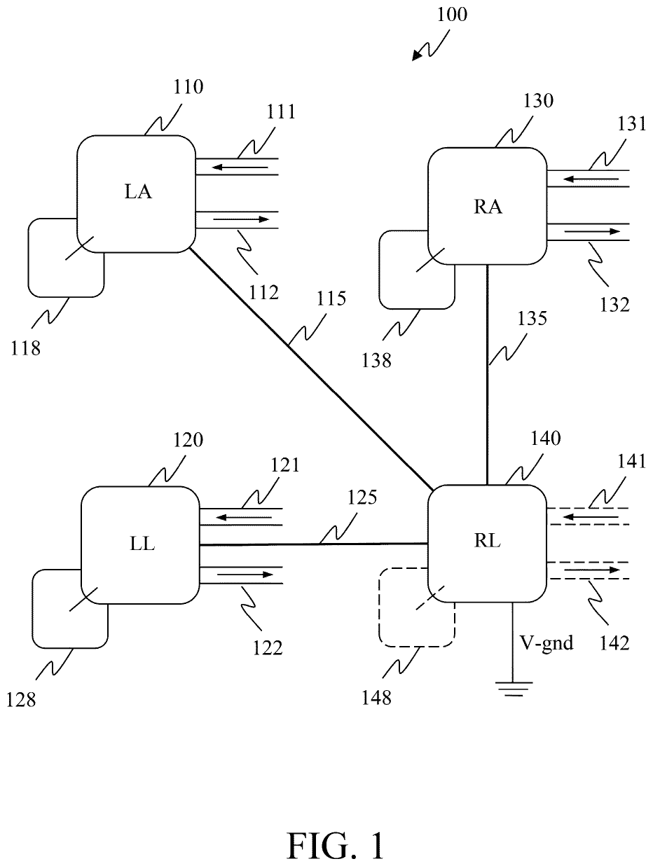

Referring to FIG. 1, in one embodiment, is a schematic representation of a set of measurement nodes for monitoring ECG signals from a subject. The measurement node set 100 is attachable to the skin of a subject for acquiring ECG pulses produced by the subject's heartbeat. The measurement node set 100 includes a measurement node 110 (e.g., left arm (LA) measurement node), a measurement node 120 (e.g., left leg (LL) measurement node), a measurement node 130 (e.g., right arm (RA) measurement node), and a measurement node 140 (e.g., right leg (RL) measurement node). Any one of these nodes may be a common node, and in this example node 140 is a common node. As discussed below, the measurement nodes 110, 120 and 130 include the necessary components for receiving the ECG pulses, converting the ECG pulses into optical ECG pulses, and communicating the optical pulses over optical fiber, thus eliminating the need for electrical ECG leads. In various configurations, the number of measurement nodes in the measurement node set 100 may vary to provide unique benefits for any particular situation or to meet application specific design requirements of various implementations, as would be apparent to one skilled in the art.

The common node 140 creates a common electrical reference for the measurement node set 100, referred to as virtual ground (V-gnd). In an embodiment, the common node 140 may also be a measurement node with the same configuration as the measurement nodes 110, 120 and 130. In this case, the common node 140 may also be used for receiving the ECG pulses, converting the ECG pulses into optical ECG pulses, and communicating the optical pulses over optical fiber. Alternatively, the common node 140 may simply create the virtual ground without the functionality of a measurement node.

Since the common node 140 creates the virtual ground, the measurement nodes 110, 120 and 130 are connected to the common node 140 through short conductive paths in relation to the subject's body length, so that they have a common ground potential without having to be electrically grounded, e.g., through long ECG leads. A short conductive path may be less than a quarter of the subject's body length, while the long ECG leads exceed the subject's body length. In particular, the measurement node 110 is connected to the common node 140 via conductive path 115, the measurement node 120 is connected to the common node 140 via conductive path 125, and the measurement node 130 is connected to the common node 140 via conductive path 135. Each of the short conductive paths 115, 125 and 135 is formed of a highly electrically conductive material, such as copper, aluminum, gold or silver, for example, and is no longer than about 12 cm, for example.

In the depicted embodiment, each of measurement nodes 110, 120 and 130 is connected to a corresponding ECG electrode that attaches to the skin of the subject at specific locations on the subject's body to acquire ECG pulses generated from the subject's heartbeat. In particular, the measurement node 110 is connected to ECG electrode 118, the measurement node 120 is connected to ECG electrode 128, and the measurement node 130 is connected to ECG electrode 138. The common node 140 is shown as optionally connected to ECG electrode 148 (indicated by dashed lines), which would occur when the common node 140 also has the functionality of a measurement node, as discussed above. The measurement nodes 110, 120 and 130 may be detachably connected to the ECG electrodes 118, 128 and 138, in which case conventional ECG electrodes may be used. For example, the measurement nodes 110, 120 and 130 may snap or clip onto respective upper surfaces (facing away from the subject's body) of the corresponding ECG electrodes 118, 128 and 138. Alternatively, the ECG electrodes 118, 128 and 138 may be physically integrated within the measurement nodes 110, 120 and 130, respectively.

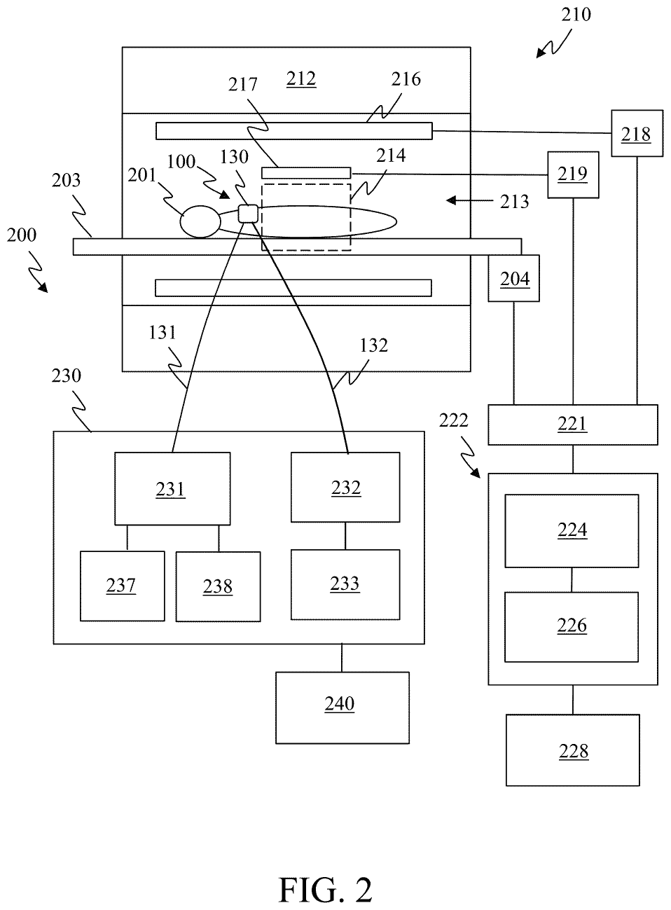

The measurement nodes 110, 120 and 130 are further configured to communicate with an ECG module (not shown), discussed below with reference to FIG. 2. Generally, the ECG module provides DC power and optionally clock signals to the measurement nodes 110, 120 and 130 via input fiber-optic cables, and processes the ECG signals provided by the measurement nodes 110, 120 and 130 via output fiber-optic cables. In particular, the measurement node 110 is connected to input fiber-optic cable 111 and output fiber-optic cable 112, the measurement node 120 is connected to input fiber-optic cable 121 and output fiber-optic cable 122, and the measurement node 130 is connected to input fiber-optic cable 131 and output fiber-optic cable 132. The common node 140 is shown as optionally connected to input fiber-optic cable 141 and output fiber-optic cable 142 (indicated by dashed lines). As mentioned above, this because the common node 140 may be configured as a measurement node to acquire ECG signals.

Referring to FIG. 2, in one embodiment, is an ECG system for monitoring ECG pulses from a subject, implemented within magnetic resonance imaging (MRI) system. Although depicted with the MRI system for purposes of explanation, it is understood that the ECG system may be implemented on its own or with any other type of medical imaging or medical testing system, without departing from the scope of the present teachings.

Referring to FIG. 2, ECG system 200 is incorporated with representative MRI system 210 in order to monitor ECG pulses of a subject 201 during an MRI procedure. The MRI system 210 may be any type of MRI system, and the following description of the MRI system 210 is intended to be illustrative and not limiting. In the depicted example, the MRI system 210 includes a magnet 212 with a bore 213. The magnet 212 may be a superconducting cylindrical magnet, for example, although use of different types of magnets is possible, such as a split cylindrical magnet and an open magnet. An imaging zone 214 is provided in the bore 213 where the magnetic field generated by operation of the magnet 212 is strong and uniform enough to perform the magnetic resonance imaging.

The subject 201 is placed on a support 203 and positioned within the bore 213 to be imaged during the MRI procedure. The support 203 may be attached to an actuator 204 (optional) configured to move the support 203, so that the subject 201 may be moved through the imaging zone 214. Accordingly, a larger portion of the subject 201 or the entire subject 201 may be imaged.

The ECG system 200 includes the measurement node set 100, discussed above. Accordingly, the ECG electrodes 118, 128 and 138 respectively corresponding to the measurement nodes 110, 120 and 130 are attached to the skin of the subject 201 in order to perform ECG monitoring during the MRI procedure. Only the measurement node 130 is shown in FIG. 2 for the sake of convenience. As discussed above, the common node 140 (not shown) creates a virtual ground, and the measurement node 130 is connected to the common node 140 by the short conductive path 135 in order to provide the common electrical reference to the measurement node 130. The other measurement nodes 110 and 120 (not shown) are likewise connected to the virtual ground provided by the common node 140, as discussed above. In an embodiment, the common node 140 is also a measurement node, and is connected to the corresponding ECG electrode 148.

The MRI system 210 includes a set of magnetic field gradient coils 216 configured to acquire magnetic resonance data for spatially encoding magnetic spins within the imaging zone 214. A magnetic field gradient coil power supply 218 supplies current to the magnetic field gradient coils 216. The current may be controlled as a function of time, and may be ramped or pulsed, for example. Although two magnetic field gradient coils 216 are shown, it is understood that additional magnetic field gradient coils may be included, e.g., to enable spatially encoding in three orthogonal spatial directions.

The MRI system 210 further includes RF coil 217 located within the bore 213. The RF coil 217 is configured to manipulate orientations of magnetic spins within the imaging zone 214, and to receive RF transmissions from spins also within the imaging zone 214. The RF coil 217 may represent dedicated transmit and receive antennas or may contain multiple transmit and receive coil elements. The RF coil 217 is shown connected to an RF transceiver 219, which transmits and receives RF signals to and from the RF coil 217 during the MRI procedure. In various configurations, the RF coil 217 and the RF transceiver 219 may be replaced by separate transmit and receive coils and separate transmitters and receivers, for example.

The actuator 204, the magnetic field gradient coil power supply 218, and the RF transceiver 219 are connected to a hardware interface 221 and a controller 222. The controller 222 includes a processor 224, memory 226, and a user interface 228. The memory 226 represents one or more non-transitory memories and/or data storage, discussed further below. The memory 226 may store pulse sequence instructions, which are executed by the processor 224 for performing the MRI procedure. The memory 226 may also include data storage for storing magnetic resonance data and/or reconstructed magnetic resonance images acquired during the MRI procedure. The hardware interface 221 enables the controller 222 to interact with, control and/or exchange data with at least the actuator 204, the magnetic field gradient coil power supply 218, and the RF transceiver 219. The hardware interface 221 may include one or more of a universal serial bus (USB), IEEE 1394 port, parallel port, IEEE 1284 port, serial port, RS-232 port, IEEE-488port, Bluetooth connection, wireless local area network connection, TCP/IP connection, Ethernet connection, control voltage interface, MIDI interface, analog input interface, and digital input interface, for example.

The processor 224 is representative of one or more processing devices and may be implemented by a general-purpose computer, a central processing unit, a computer processor, a microprocessor, a microcontroller, field programmable gate arrays (FPGAs), application specific integrated circuits (ASICs), a state machine, programmable logic device, or combinations thereof, using any combination of hardware, software, firmware, hard-wired logic circuits, or combinations thereof. A processor may also refer to a collection of processors within a single computer system or distributed among multiple computer systems, such as in a cloud-based or other multi-site application.

The memory 226 may be implemented by any number, type and combination of random-access memory (RAM) and read-only memory (ROM), for example, and may store various types of information, such as software algorithms, artificial intelligence (AI) machine learning models, and computer programs, all of which are executable by the processor 224. The various types of ROM and RAM may include any number, type and combination of non-transitory computer readable storage media, such as a disk drive, flash memory, an electrically programmable read-only memory (EPROM), an electrically erasable and programmable read-only memory (EEPROM), registers, a hard disk, a removable disk, tape, compact disk read only memory (CD-ROM), digital versatile disk (DVD), floppy disk, Blu-ray disk, a universal serial bus (USB) drive, or any other form of storage medium known in the art. As used herein, the term non-transitory is to be interpreted not as an eternal characteristic of a state, but as a characteristic of a state that will last for a period. The term non-transitory specifically disavows fleeting characteristics such as characteristics of a carrier wave or signal or other forms that exist only transitorily in any place at any time.

The user interface 228 enables a user or operator to interact with the controller 222, receiving input from the operator to be received by the processor 224 and providing output to the user from the processor 224. That is, the user interface 228 may provide information or data to the operator and/or receive information or data from the operator. The display of data or information on a display or a graphical user interface is an example of providing information to the operator. The receiving of data through a keyboard, mouse, trackball, touchpad, pointing stick, graphics tablet, joystick, gamepad, webcam, headset, pedals, wired glove, remote control, and accelerometer are all examples of components of the user interface 228 which enable the receiving of information or data from the operator.

In addition to the measurement node set 100, the ECG system 200 further includes an ECG module 230 and an output 240. In the depicted embodiment, the ECG module 230 includes an optical modulator 231, an optical demodulator 232, and a processor 233. The optical modulator 231 is configured to receive a clock signal from a clock 237 and a light signal from a light source 238, to modulate the light signal and the clock signal using any compatible modulation technique, and to output a modulated optical signal with an embedded clock signal to the measurement nodes 110, 120 and 130 via the respective input fiber-optic cables 111, 121 and 131, respectively. The light source 238 may be a laser or a light emitting diode (LED), for example. In an embodiment, the optical modulator 231 may provide a pulse width modulated (PWM) optical signal with an embedded clock signal, which may be embedded via light pulses, for example. Alternatively, the optical modulator 231 may provide a frequency modulated or amplitude modulated optical signal with the embedded clock signal. The frequencies and/or widths of the light pulses in the PWM optical signal and the embedded clock signal, for example, may be adjusted to suit the MRI scanning environment. For example, certain frequencies must be avoided as to not interfere with the MR scanned image. A tunable configuration of the ECG module 230 allows all frequencies to be selected or avoided.

The optical demodulator 232 is configured to receive optical ECG signals from the measurement nodes 110, 120 and 130 via the respective output fiber-optic cables 112, 122 and 132, respectively, and to convert the ECG signals into corresponding electrical signals. The processor 233 is configured to execute instructions stored in a non-transitory memory (not shown) for processing the electrical signals to provide a corresponding ECG wave to the output 240. The instructions may further cause the processor 233 to define characteristics of the ECG signals, such as the QRS complex, average beat, heart rate variability, RR interval, PR interval, and pulse rate, for example. The memory may be one or more non-transitory memories and/or data storage, as described above with reference to the memory 226.

The processor 233 is representative of one or more processing devices, and may be implemented by a general-purpose computer, a central processing unit, a computer processor, a microprocessor, a microcontroller, FPGAs, ASICs, a state machine, programmable logic device, or combinations thereof, using any combination of hardware, software, firmware, hard-wired logic circuits, or combinations thereof. A processor may also refer to a collection of processors within a single computer system or distributed among multiple computer systems, such as in a cloud-based or other multi-site application.

The output 240 may include any type of visual manifestation of the ECG traces. For example, the output 240 may include a display for displaying the ECG wave, such as a computer monitor, a television, a liquid crystal display (LCD), an organic light emitting diode (OLED) display, a flat panel display, a solid-state display, or a cathode ray tube (CRT) display, a touch screen or an electronic whiteboard, for example. Alternatively, or in addition, the output 240 may include a printer, such as a thermal printer or an inkjet printer, for example, for printing the ECG wave. ECG wave may be displayed and/or printed together with textual and/or graphical information that classifies and/or interprets the ECG wave.

In the depicted embodiment, the measurement nodes 110, 120 and 130 are physically connected to the ECG module 230 via the input fiber-optic cables 111, 121 and 131 and the output fiber-optic cables 112, 122 and 132, respectively. However, in an alternative embodiment, the measurement nodes 110, 120 and 130 may be connected to a transceiver and antenna (not shown) via the input fiber-optic cables 111, 121 and 131 and the output fiber-optic cables 112, 122 and 132, respectively, where the transceiver is configured to communicate wirelessly with the ECG module 230. In this case, the ECG module 230 would likewise include a transceiver and antenna (not shown) for sending the DC power and clock signals and receiving the ECG signals.

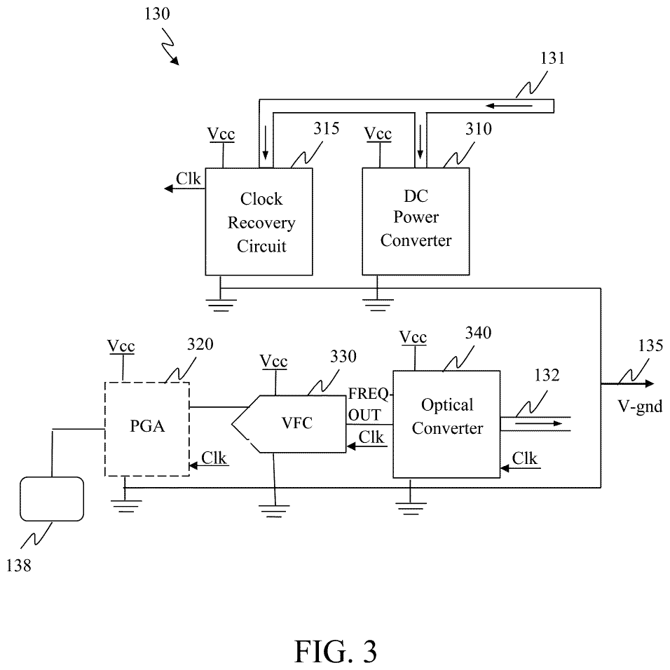

As discussed above, the measurement nodes 110, 120 and 130 have the same configuration. In various implementations, the common node 140 may also have the same configuration as the measurement nodes 110, 120 and 130 (except for the conductive paths). FIG. 3 is a simplified block diagram showing an illustrative measurement node for monitoring ECG signals from a subject, according to a representative embodiment. In particular, FIG. 3 shows the measurement node 130 as being representative of all the measurement nodes, for purposes of illustration.

Referring to FIG. 3, the measurement node 130 includes a DC power converter 310 and a clock recovery circuit 315, which are connected to the input fiber-optic cable 131. The DC power converter 310 is configured to receive the modulated optical signal from the optical modulator 231 of the ECG module 230 via the input fiber-optic cable 131, and to convert the modulated optical signal to a corresponding electrical signal. By converting the modulated optical signal to the electrical signal, the DC power converter 310 recovers DC power embedded within the modulated optical signal. For example, when the modulated optical signal is a PWM optical signal, the magnitude of the DC power is indicated by the frequency and/or widths of the light pulses. The DC power converter 310 may be a photovoltaic cell, for example, which converts optical signals directly into electrical signals using photovoltaic effect.

According to an embodiment, the clock recovery circuit 315 recovers the embedded clock signal from the modulated optical signal. The clock recovery circuit 315 may be an edge detector, phase detector or a frequency detector, for example. The detectors of the clock recovery circuit depend on how the clock is optically encoded, as is known in the art. Recovery of the DC power and the embedded clock signal may be performed in any order or simultaneously. The DC power converter 310 outputs the DC power (Vcc) and the clock recovery circuit 315 outputs the recovered clock signal (Clk) to other components of the measurement node 130, discussed below.

The measurement node 130 is shown connected to the ECG electrode 138, which is attached to the skin of the subject 201, to receive small analog ECG pulses, which may be in the μV to mV ranges. The measurement node 130 provides an analog front end for the ECG electrode 138, including an optional programmable gain amplifier (PGA) 320 (indicated by dashed lines) and voltage-to-frequency converter (VFC) 330, as well as an optical converter 340. As shown, each of the PGA 320, the VFC 330, and the optical converter 340 receive the DC power (Vcc) from the DC power converter 310. Additionally, optionally each of the PGA 320, the VFC 330, and the optical converter 340 receive the recovered clock signal (Clk) from the clock recovery circuit 315. Accordingly, the PGA 320, the VFC 330, and the optical converter 340 are powered without an electrical power source using the DC power (Vcc) and are optionally synchronized with one another using the recovered clock signal (Clk).

The PGA 320 receives the analog ECG pulses from the ECG electrode 138, which are electrical signals. The VFC 330 converts the ECG pulses into the frequency domain, and this digital signal (FREQ-OUT) is used to modulate a set of frequencies at each ECG measurement node. This minimizes signal degradation from noise sources and avoids interference with the MRI when implemented in an MRI environment. Additionally, using the VFC can eliminate the need for a synchronized distributed clock within the ECG system. The VFC also encodes the analog ECG pulse at very low power, thereby reducing the power needs of the ECG system.

According to an embodiment, the VFC 330 of each of the measurement nodes 110, 120 and 130 and the common node 140 can frequency-encode the ECG pulses received from the respective ECG electrode at a different frequency. Accordingly, the ECG system can be configured to recognize the different frequencies of the frequency-encoded ECG pulses and thus determine which node is transmitting or transmitted information based on the recognized frequency. Additionally, the VFC 330 can be designed or selected to encode ECG pulses at a frequency that will not interfere with the MRI or other environment in which the ECG system is implemented.

The optical converter 340 receives the frequency modulated data stream from the VFC 330 and convert it to an optical ECG signal. The optical converter 340 may be a laser or an LED light source, for example. The optical converter 340 outputs the optical ECG signals to the optical demodulator 232 of the ECG module 230 via the output fiber-optic cable 132.

The grounds of each of the DC power converter 310, the clock recovery circuit 315, the PGA 320, the VFC 330, and the optical converter 340 are connected to the virtual ground (V-gnd) created by the common node 140 via the conductive path 135. Accordingly, the DC power and clock recovery circuit 315, the PGA 320, the VFC 330, and the optical converter 340 are grounded to a common potential, along with the components of the other measurement nodes (e.g., measurement nodes 110, 120), without having to be electrically grounded elsewhere in the ECG system 200. The recovered DC power and the virtual grounding of the measurement node 130 eliminate the need for electrical leads connecting the measurement node 130 to an external power source and ground.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A system for acquiring electrocardiogram (ECG) pulses from a subject, the system comprising:
a virtual ground; and
a plurality of measurement nodes connectable to a plurality of corresponding ECG electrodes, wherein each of the plurality of corresponding ECG electrodes are attachable to the subject, wherein the plurality of measurement nodes are connected to the virtual ground, and wherein each of the plurality of measurement nodes comprises:
a voltage-to-frequency converter (VFC) configured to convert an ECG signal from the corresponding ECG electrode to a frequency signal;
an optical converter configured to convert the frequency signal from the VFC to an optical signal, and to output the optical signal via an output fiber-optic cable; and
a DC power converter configured to receive a modulated optical signal via an input fiber-optic cable, to recover DC power from the modulated optical signal, and to supply the DC power to at least the VFC and the optical converter,
wherein no electrical leads are connected to the plurality of measurement nodes.

2. The system of claim 1, wherein the modulated optical signal comprises an embedded clock signal, and wherein the system further comprises, for each of the plurality of measurement nodes, a clock recovery circuit configured to receive the modulated optical signal with the embedded clock signal via the input fiber-optic cable, to recover the embedded clock signal from the modulated optical signal, and to supply the recovered clock signal to at least the VFC and the optical converter for synchronization.

3. The system of claim 1, further comprising:
an ECG module configured to provide the modulated optical signal to the plurality of measurement nodes via the input fiber-optic cables respectively, to receive the optical signals from the plurality of measurement nodes via the output fiber-optic cables respectively, and to convert the optical signals to the ECG pulses.

4. The system of claim 1, wherein the plurality of measurement nodes comprise a left arm (LA) measurement node, a right arm (RA) measurement node, and a left leg (LL) measurement node, and a right leg (RL) measurement node.

5. The system of claim 1, wherein the DC power converter of each measurement node of the plurality of measurement nodes comprises a photovoltaic cell.

6. The system of claim 1, wherein each measurement node of the plurality of measurement nodes further comprises:
a programmable gain amplifier (PGA) connected to an input of the VFC, and configured to amplify the ECG signal.

7. The system of claim 1, further comprising:
a monitor configured to display the ECG pulses output by the ECG module.

8. The system of claim 1, wherein the subject is positioned within a magnet resonance imaging (MRI) bore while the ECG pulses are acquired.

9. The system of claim 1, wherein the modulated optical signal comprises a pulse width modulated (PWM) optical signal.

10. The system of claim 1, wherein the modulated optical signal comprises a frequency modulated or amplitude modulated optical signal.

11. The system of claim 1, wherein the VFC of each measurement node is configured to convert the ECG signal to a different frequency signal relative to every other measurement node.

12. A system for acquiring electrocardiogram (ECG) pulses from a subject, the system comprising:
a virtual ground;
a plurality of measurement nodes connectable to a plurality of corresponding ECG electrodes, wherein each of the plurality of corresponding ECG electrodes are attachable to the subject and comprise a left arm (LA) measurement node, a right arm (RA) measurement node, and a left leg (LL) measurement node, and a right leg (RL) measurement node, wherein the plurality of measurement nodes are connected to the virtual ground, and wherein each of the plurality of measurement nodes comprises:
a programmable gain amplifier (PGA) configured to amplify the ECG signal;
a voltage-to-frequency converter (VFC) configured to convert an ECG signal to a frequency signal;
an optical converter configured to convert the frequency signal from the VFC to an optical signal, and to output the optical signal via an output fiber-optic cable; and
a DC power converter configured to receive a modulated optical signal via an input fiber-optic cable, to recover DC power from the modulated optical signal, and to supply the DC power to at least the VFC and the optical converter;
an ECG module configured to provide the modulated optical signal to the plurality of measurement nodes via the input fiber-optic cables respectively, to receive the optical signals from the plurality of measurement nodes via the output fiber-optic cables respectively, and to convert the optical signals to the ECG pulses, wherein no electrical leads are connected between the ECG module and the plurality of measurement nodes; and
a monitor configured to display the ECG pulses output by the ECG module.

13. The system of claim 12, wherein the modulated optical signal comprises an embedded clock signal, and wherein the system further comprises, for each of the plurality of measurement nodes, a clock recovery circuit configured to receive the modulated optical signal with the embedded clock signal via the input fiber-optic cable, to recover the embedded clock signal from the modulated optical signal, and to supply the recovered clock signal to at least the VFC and the optical converter for synchronization.

14. The system of claim 12, wherein the subject is positioned within a magnet resonance imaging (MRI) bore while the ECG pulses are acquired.

15. The system of claim 12, wherein the VFC of each measurement node is configured to convert the ECG signal to a different frequency signal relative to every other measurement node.
